(19) United States
(12) Patent Application Publication
(10) Pub. No.: US 2010/0197943 A1
(43) Pub. Date: Aug. 5, 2010

(54) Biodiesel production unit and biodiesel compositions

(76) Inventors: Bryon Tarbet, Highland, UT (US); Kenneth Tarbet, Snowflake, AZ (US)

Correspondence Address: KENNETH H. TARBET, 585 South 2nd West, Snowflake, AZ, 85937, US

(21) Appl. No.: 12/365,054

(22) Filed: Feb. 3, 2009

(51) Int. Cl. C07D 309/00; C07D 307/02; C07C 69/00

(52) U.S. Cl. 549/356; 549/505; 560/129

(57) ABSTRACT

A biodiesel composition and method are disclosed and claimed whereby a biodiesel fuel is produced without glycerin waste and which has a could point below 32° F.

This application claims priority filing date of US provisional application Ser. No. 60/909,908 filed Apr. 3, 2007, the contents of which are incorporated by reference in their entirety.

### FIELD OF THE INVENTION

The invention primarily relates to systems and methods for manufacturing and processing fuels and, more particularly, relates to systems and methods for manufacturing and processing biodiesel fuels without the use of a water-washing step. The present system, methods and composition relate to the conversion of the produced glycerin to tri-ethers, useful as additives and biodiesel modifiers. The present composition relates to the generation of unique molecular configurations providing a winterized biodiesel and a biofuel of improved properties.

### BACKGROUND OF THE INVENTION

Biodiesel is the name for a variety of ester-based oxygenated fuels made from vegetable oils, fats, greases, or other sources of triglycerides. It is a nontoxic and biodegradable substitute and supplement for petroleum diesel. Even in blends as low as 20% biodiesel to 80% petroleum diesel (B20), biodiesel can substantially reduce the emission levels and toxicity of diesel exhaust. Biodiesel has been designated as an alternative fuel by the United States Department of Energy and the United States Department of Transportation, and is registered with the United States Environmental Protection Agency as a fuel and fuel additive. It can be used in any diesel engine, without the need for mechanical alterations, and is compatible with existing petroleum distribution infrastructure.

As reported in “Biodiesel: On the Road to Fueling the Future,” (National Biodiesel Board 2001), the disclosure and subject matter of which is hereby incorporated by reference in its entirety, most biodiesel is produced by the process of acid or base catalyzed transesterification. The transesterification process is a low temperature (150° F.), low pressure (20 psi) reaction having a high conversion factor (e.g. 98%) with minimal side reactions and reaction time.

A fat or oil is reacted with an alcohol (such as methanol or ethanol) in the presence of a catalyst to produce glycerin and alkyl esters, the latter of which comprises biodiesel. Traditionally, the alcohol is charged in an excess stoichiometric amount to drive the reaction and is recovered for reuse. The catalyst is typically sodium or potassium hydroxide which is mixed with the alcohol prior to the transesterification reaction. The biodiesel is separated from the glycerin. Variations, improvements, and modifications of this general process are described in several patents, including U.S. Pat. No. 5,424,467 entitled “Method for Purifying Alcohol Esters,” issued to Bam et al. on Jun. 13, 1995.

Conventional biodiesel production systems are based upon large, fixed base plants which require expensive capitalization and on site construction. For example, in order to generate an economically viable amount of biodiesel product, a conventional biodiesel plant contains large, batch-type reactors, large separation units (e.g., decanters, centrifuges, clarifiers), and distillation columns as tall as 50 to 200 feet or more. As a result, current biodiesel production is limited to discrete locations where fixed plants may be constructed. This results in inefficiencies that may otherwise be obtained by locating a plant near a source of raw materials, or near an end user of the biodiesel product. Further, the conventional process relies upon batch processing, in which the transesterification reaction proceeds in at least two multi-hour stages and in which the separation processes cannot be performed continuously. Moreover, the conventional process relies on at least one washing of the crude biodiesel product with water.

### SUMMARY OF THE INVENTION

The present invention was created in order to solve the above problems associated with large, fixed base plants that are conventionally used to produce biodiesel, other fuels, and other products. An object of the present invention is to provide a modular production system capable of producing small to large quantities of biodiesel. In another embodiment of the present invention is to provide molecular blends of biodiesel that have a cloud point below 32° F. In yet another embodiment of the present invention is provided the separate transformation of the waste glycerin into a fuel additive. In a still further embodiment of the present invention is directed to selectively combining feedstock oils providing a blend of triglycerides of varied carbon chain lengths and double bonds, which result in a biodiesel having a gel point below 32° F. In another aspect of the invention is provided a system for producing biodiesel without a step wherein the biodiesel is washed with water.

In the preferred embodiments, the described production systems are capable of producing biodiesel at a rate of about 1 million to about 25 million gallons per month. While the systems and methods are described herein in specific relation to the production of biodiesel, those of ordinary skill in the art will recognize that the advantages obtained by these systems and methods may be applied to the production of other fuels and other products as well.

In a first aspect, a preferred biodiesel manufacturing and processing system and method includes a preassembled, modular production unit that, in a preferred form, includes the following system components: a. a mixing unit; b. a reaction chamber; c. a separator unit; d. a filtering unit.

The above components of the modular production unit are preferably incorporated onto a single platform, such as a skid mount, or into a housing, such as a standard ISO Intermodal Shipping Container, such that the system is easily shipped or transferred to a remote site by either truck, rail, ship, or other means of transportation. Thus, each component and the overall system are designed to address the constraints of limited space availability, while at the same time providing for maximum throughput and processing of the widest variety of feedstocks into fuel products.

The basic biodiesel reaction of converting organic oils into alkyl esters (biodiesel) and glycerin involves the reaction of a raw oil with an alcohol (typically methanol or ethanol, although most alcohols can be made to work) and a catalyst (typically sodium hydroxide or potassium hydroxide). The equipment used for this process generally consists of large reactor tanks with paddle type mixers in steam jacketed tanks. The alcohol and the catalyst are mixed first, then the alcohol/catalyst mixture is mixed with the raw oil heated to about 140°-160° F. and allowed to react over a 4 to 8 hour time.

One challenge for the modular production unit of the present invention was to create an alcohol/catalyst mixer and a separation reactor that could both react and separate waste within the space constraints of the modular unit. This is accomplished according to a preferred embodiment of the present invention by replacing the conventional heated tank with one or more static mixing devices as further depicted in the figures. A traditional pump facilitates circulation of reactants and their passage through the reaction chamber, yet an elevated mixing tank could rely on gravity and function according to the present disclosure. A tank for each reactant may be employed, where the proper amount of each reactant is metered into the reaction chamber. Where space requirements or desire demand the use of one tank, the reactants are combined therein and are drawn from a cone-shaped bottom portion of the tank. This creates a central vortex within the reaction tank that allows rapid and complete mixing of the reactants prior to entering the reaction chamber. Similar, yet standard pumps are also used for filling and emptying the alcohol/catalyst mixer and the separation reactor. A venturi valve or similar device as is known in the art, couples the alcohol/catalyst mixer with the separation reactor to allow the constant introduction of the correct proportion of alcohol/catalyst to raw oil.

Once the reactants pass through the reaction chamber the crude product begins to separate into biodiesel and glycerin.

An additional aspect of the present invention involves the application of electrostatic energy to facilitate the separation of glycerin and alkyl-ester product. This is performed by inserting in the flow of the crude material, both (+) positive and negative (−) terminals from a power source. The electrical current should be greater than 400 volts with less than about 1 amp, but preferably greater than 1000 volts and less than 0.5 amps. This technique is most useful if implemented right before and/or as the crude material enters a tank or other storage container as shown in Figure III. The crude material enters from the top or upper entrance, the voltage is provided through electrodes continually immersed in the fluid. As the glycerin falls to the bottom of the vessel it is allowed to flow out the exit port as shown. The clean ester/biodiesel product is extracted through the upper exit. Placing this pre-cleaning device in the system accomplishes several important events.

All chemical reactions operate according to one or more systems in equilibrium. The equilibrium between the formation of product versus the formation of starting materials, the reverse reaction, is governed by le chatelier's principle. This principle provides that the equilibrium will favor product formation when there is an excess or a pressure is placed on the starting materials. In a like manner the formation of product will be favored when a paucity of product is found. In the current reaction, starting materials are the oil (triglyceride) and alcohol with products being the alcohol-ester and glycerin. The reaction is influenced to form product more quickly and efficiently when the amount of starting alcohol is increased and/or the amount of glycerin is reduced. Therefore, using the above electrostatic pre-cleaning system will remove substantial amounts of glycerin thereby promoting the forward reaction and formation of the desired alcohol ester.

A further embodiment of the present invention uses solid acid or super acid components which may be part of a particle having magnetic poles and which may be very small, even nanoparticles. Under this paradigm, the reaction is acid catalyzed; yet still providing biodiesel products according to the present claims.

According to convention and practice known in the art, as the glycerin is formed the reaction approaches equilibrium and begins to slow, and eventually stops, even before all of the raw oil has been reacted. Moreover, the conventional practice is to allow the product to reach saturation, draw off the glycerin (along with any excess alcohol), and then re-commence the transesterification reaction using the remaining mixture of raw oil, alcohol and biodiesel. This process generally takes four to six hours and significant excess alcohol. By using the alcohol/catalyst mixer the reaction chamber and the separation reactors and electrostatic pre-seperators in the manner provided by the present invention, the process is sped up to occur within minutes without the need for excess alcohol.

Another aspect of the present invention is directed to a compound according to compound I.

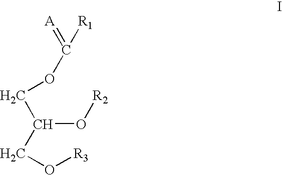

Where R₁ is an alkyl chain of between 10 to 25 carbons with one or more than one double bonds and A is a hetero-atom such as O, N or S or is such an atom as part of another moiety such as a ring, such as furan or pyran.

R₂ and R₃ are independently selected from the list comprising H, C₁₀-C₂₀ saturated or unsaturated ethers, including glycol ethers.

The glycerin may further be collected and modified into a diesel additive according to formula II.

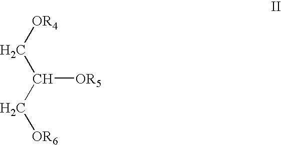

Where R₄, R₅ and R₆ are independently selected from the group comprising C₁ to C₁₅ substituted or un-substituted and saturated or unsaturated, straight or branched, alkyl or heteroalkyl moieties. Furthermore, R₄, R₅ and R₆ are selected from substituted or un substituted alkyl or heteroalkyl rings such as benzene, toluene, furan and pyran rings.

Although as glycerin forms it will naturally fall to the bottom, the present invention includes an electrostatic system whereby a current of about 1000 or more volts and about 1 or less milliamps is applied. The result is the increased rate of glycerin separation. This, when combined with the “pause” tank according to the flow diagram allows for maximum separation of glycerin, thereby further improving reaction efficiency.

Importantly, an analogous process is used to transform glycerin back into biofuel as depicted in equation I.

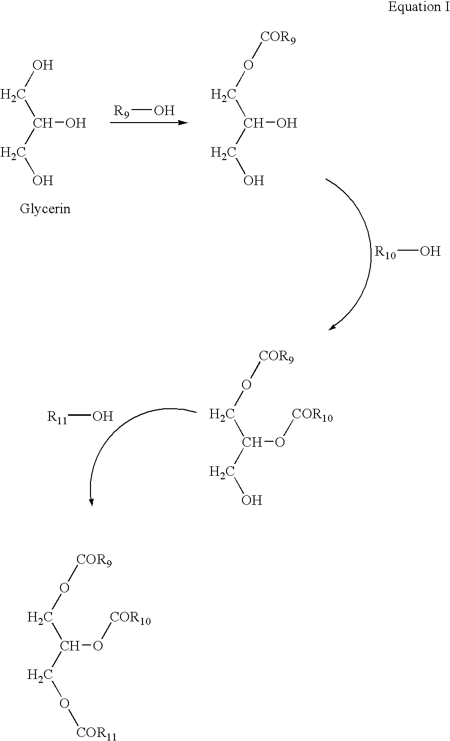

Where R₉-R₁₁ are independently selected from the group comprising saturated or unsaturated alkyl chains of five to fifteen carbons, which may be substituted with additional carbon chains or heteroatoms including heteroatoms in chains of either linear or in rings. In this manner, the glycerin, which has heretofore been seen as a waste product is now a useful and even desired product.

As glycerin is formed it naturally drops to the cone-shaped bottom of the separation reactor. As part of the recirculation of the reactants, the reactants are run through an array, preferably three, of serial centrifuges to separate the glycerin from the reactants and biodiesel. The present system may be performed in a continuous mode or in a continuous batch mode as will be further provided in the examples. Yet in brief, under continuous mode, reactants are pulled together at a metered amount and into a common tube which feeds into the reaction chamber wherein the reactants are exposed to the ultrisonification energy whereby transesterification is caused. The crude mixture then passes through a centrifuge where the biodiesel and glycerin are separated. Under a continuous batch mode system, the reactants are combined in a single reaction vessel and pumped through a static mixer, as known in the art, and into the reaction chamber. The reactants are exposed to the ultrasonication energy whereby transesterification occurs. The reaction mixture is recycled through the storage tank, in a circulation fashion which includes the reaction chamber. Although there are reasons for adopting either system and reaction paradigm, they are both encompassed by the present invention and claims.

In the preferred embodiment, the alcohol/catalyst mixer and separation reactor comprise tanks made of epoxy coated steel, or entirely in stainless, or some combination of the two. The number and size of pumps and static mixers can be varied to optimize the creation of a mixing vortex, and the direction angle can be varied in the same manner, and/or optimized for Coriolis efficiency (counter clockwise in the Northern Hemisphere, and clockwise in the Southern Hemisphere). Further embodiments include embedded instrumentation for monitoring temperature, PH, flow rates and volumes, and fill levels.

Although several advantages are obtained by providing the systems and methods described herein in a self-contained, modular production unit, those skilled in the art will recognize that one or more of the described system components may be provided in a scaled up form for use in a fixed base, nonmodular configuration to obtain the other advantages provided by those components.

In a second aspect, the modular production unit described above is combined with additional fixed and/or relocatable system components in a biodiesel processing plant. In a preferred form, the plant is provided with components and functionality to provide raw materials processing and finished biodiesel product processing. In particular, the raw materials processing includes filtering and separation functionality to remove waste and particulate matter from recycled triglycerides starting materials. Further, the finished biodiesel product processing includes filtering and separation functionality to reprocess the glycerin into alkyl, heteroalkyl, aryl or similar triethers. Modified glycerin in this manner functions as a gel-point depressant and a CETANE enhancer.

In a particularly preferred form, the raw materials processing and finished biodiesel product processing systems are co-located on a single or double transportable platform, such as a skid mount, or in a transportable housing, such as a standard shipping container. In this manner, similar to the modular production unit described herein, the raw materials and finished product processing systems may be relocated to a desired site.

The biodiesel processing plant is preferably provided with additional optional components, including storage tanks, spill areas, and/or other components that may provide auxiliary functionality to the plant.

The systems, methods, and apparatus of the present invention will be better understood by reference to the Detailed Description in connection with the Drawings.

### BRIEF DESCRIPTION OF THE DRAWINGS

FIG. 1 is a flow diagram showing a process for producing biodiesel according to the present invention.

FIG. 2 is a schematic diagram illustrating a method for providing a heating jacket accordance to the present invention.

FIG. 3 is a schematic diagram illustrating a preferred biodiesel manufacturing plant.

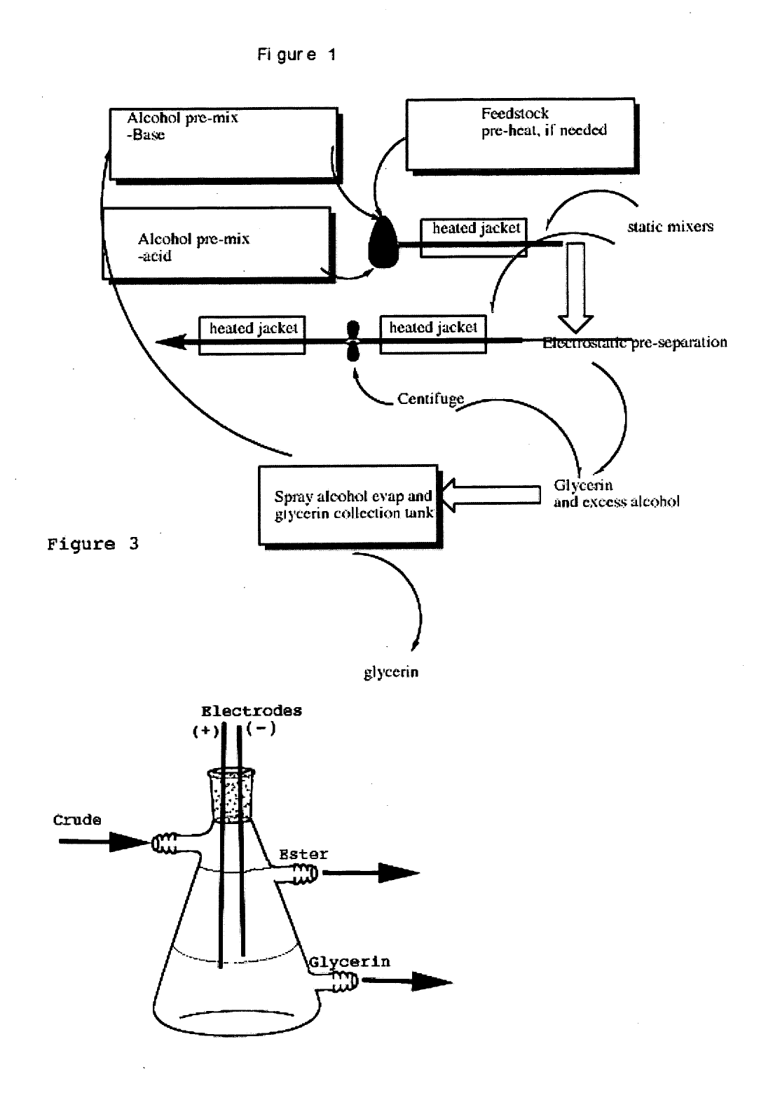

### DETAILED DESCRIPTION OF THE PREFERRED EMBODIMENTS

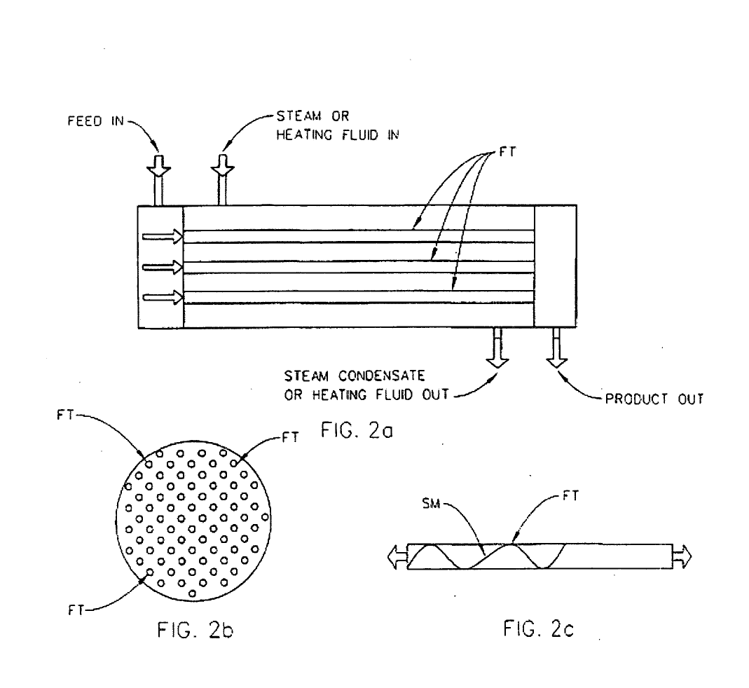

A preferred embodiment of the biodiesel manufacturing and processing systems and methods of the present invention will be described in reference to the drawings. Turning first to FIGS. 2 and 3, the present invention provides a preassembled, modular production unit that, in a preferred form, includes the following system components:

a mixing unit;

a reaction chamber;

a separator unit; and

a filtering unit.

The above components are preferably incorporated onto a single or double platform, such as a skid mount, or into a housing, such as a standard ISO Intermodal Shipping Container, such that the system is easily shipped or transferred to a remote site by either truck, rail, ship, or other means of transportation, or operated in place while situated on a truck trailer, rail car, or ship. Thus, each component and the overall system are designed to address the constraints of limited space availability, while at the same time providing for maximum throughput and processing of the widest variety of feedstocks into fuel products.

The preferred mixing unit is an alcohol/catalyst mixer that receives the alcohol and catalyst as feeds and mixes the two prior to supplying the mixture to the reaction chamber. In the preferred embodiment, the alcohol is methanol, and the catalyst is sodium hydroxide, although those of skill in the art will recognize that other alcohols and catalysts are suitable for producing biodiesel fuel. Indeed, in other preferred embodiments a mixture of alcohols is used to provide a biodiesel blend having a cloud point below 32° F. In the preferred embodiment, the alcohol/catalyst mixer comprises a 300 gallon mixing tank having a cone shaped bottom section. The tank may includes a cone-shaped bottom portion and a drain located at the bottom. When used as a mixing tank the tank advantageously includes mixing looper jets that function in a manner described below. One advantage of the cone-shaped bottom is that it allows the catalyst, which is typically in solid granular or flake form, to fall to the bottom and then be continuously recirculated and mixed until it goes into solution with the alcohol. The size of the alcohol/catalyst mixer is sufficient to produce the volume of mixed alcohol and catalyst.

The reaction chamber receives the alcohol/catalyst mixture from the mixing unit as a second feed, and the triglyceride source as a first feed, and the ultrisonification device causes the transesterification reaction to occur. After sufficient completion of the transesterification reaction, the reactor unit outputs one or more streams comprising the reaction products of the transesterification reaction, namely, biodiesel fuel (alkyl esters) and glycerin.

The separation tanks of the preferred embodiment are constructed and operate in a manner different from the batch processing reactor tanks used in conventional biodiesel manufacturing processes. The conventional batch reactors are uniform cylinders that use paddle mixers or other similar mixing. The separation tanks employed in the preferred embodiment, on the other hand, are constructed having a cone-shaped bottom equipped with a drain to facilitate removal of the glycerin phase during the transesterification process in order to provide a continuous process, unlike the prior art. The cone-shaped bottom portion and drain provided on the preferred reaction chamber facilitate this process.

The reaction chamber is further operatively connected to one or more pumps (e.g., centrifugal or gear driven) that recirculate the reaction materials through the jets to provide mixing. The jets are oriented such that, in combination with the drain at the cone-shaped bottom of the tank, a vortex is created within the reaction chamber to facilitate mixing of the materials to help drive the transesterification reaction, thereby eliminating the need for mixing paddles.

In the preferred method of operation, the separation reactor tanks are operated in a continuous mode, rather than a batch mode that is used in conventional biodiesel manufacturing processes.

For example, the conventional biodiesel manufacturing process employs a first stage reaction of approximately four hours at 140° to 160° F., after which the glycerin reaction product is drawn off, additional alcohol and catalyst are added, and a second stage reaction is conducted for approximately two additional hours, also at 140° to 160° F.

In the preferred mode of operation of the present invention, the transesterification reaction is driven to completion in a single stage within the reaction chamber, during which the reaction product streams are continuously drawn off for the separation processes described below. A complete conversion of triglyceride to biodiesel is accomplished at the rate of 2 liters per minuet.

The conventional biodiesel manufacturing process relies upon two or more distinct batch operations, whereas the system and method of the present invention advantageously provides for continuous removal of the glycerin as it is formed and for reintroduction of the alcohol/catalyst mixture, where needed.

The separation component comprises one or more components that are capable of separating the reaction products of the transesterification reaction. In the preferred embodiment, these separation components comprise an array of centrifuge units, preferably three in number, connected in series. The first centrifuge unit receives the output stream from the reactor unit, comprising the glycerin and alkyl esters along with unreacted alcohol. The first centrifuge unit separates the glycerin by-product and unreacted alcohol from the alkyl esters (biodiesel). The glycerin and alcohol are initially stored in a first holding tank, and ultimately fed to the glycerin processing reactors to produce the triethers of the present invention. The alkyl esters cut from the first centrifuge unit are then fed as an input to the second centrifuge unit, and perhaps the third as purity demands.

The modular production unit may be made entirely energy self-sufficient by operating a proper generator with biodiesel produced by the modular production unit, thus allowing the modular production unit to operate in remote locations or areas where power service is expensive and/or unreliable.

The modular production unit is preferably incorporated onto a single or double platform, such as a skid mount, or into a housing, such as a shipping container, such that the system is easily shipped or transferred to a remote site by either truck, rail, ship, or other means of transportation, or operated in place on a truck trailer, rail car, or ship. In a particularly preferred form, the modular production unit is housed in a standard ISO Intermodal Shipping Container having dimensions of 8′×8′×40′. Thus, the components associated with this preferred form of the modular production unit are of a size and shape that may be accommodated within the space limitations of the platform or housing.

Although several advantages are obtained by providing the systems and methods described herein in a self-contained, modular production unit, those skilled in the art will recognize that one or more of the described system components may be provided in a scaled up form for use in a fixed base, nonmodular configuration to obtain the other advantages provided by those components.

In the preferred embodiment, the raw materials processing is performed by a pair of settling tanks and a coalescing basket filter. The preferred raw materials processing also, optionally, includes a hot box member described in more detail below. The preferred triglycerides feedstock is any organic fat or oil, including virgin vegetable oils such as soy, canola or cottonseed, as well as recycled oils, such as used fryer oil and grease trap materials, or animal fats, such as lard or beef tallow. Many of these materials, particularly the recycled oils, will have impurities, including coarse particulates and water. The water impurities may be in the form of bulk water, entrained water, or microemulsions. The triglycerides feedstock is fed as an input to the coalescing basket filter, where the feedstock is filtered to remove particulates and/or water. The filtered feedstock is routed to one of the settling tanks, where it is stored until needed as feed to the reactor units of the modular production unit. The settling tanks are preferably heated (e.g., to at least 120° F.) and are provided with cone-shaped bottom portions and drains to promote settling and facilitate removal of waste and particulates. Such waste and particulates are advantageously removed by directing the output flow from the bottom drain through an additional coalescing basket filter of finer mesh prior to being transferred to the separation reactor tanks.

Additionally, the settling tanks may be optionally fitted with one or more metering pumps for delivering acid (such as phosphoric acid or sulfuric acid) which, in combination with heat, assists in breaking up emulsions (particularly in grease trap material) and facilitates the phase separation of the water and fats/oils/greases. Once the water is drained off, additional acid may be added to convert free fatty acids to fatty acids that can be more easily converted into alkyl esters. The acidified fats/oils/greases can then be reacted with alcohol as a first step in the separation reactors (i.e. acid catalysis) followed by a reaction with the alcohol/catalyst mixture (i.e. base catalysis) to more efficiently produce alkyl esters from high free fatty acid feedstocks such as grease trap materials.

The automated filter system serves as a fail-safe to prevent flow of biodiesel product that contains greater than the maximum amount of water desired, and preferably comprises a combination of a salt filter, a coalescing filter, a clarification filter, and a gel filter. In a preferred embodiment, the filtering system is automated to sense water build up in the first stage salt dryer and the second stage coalescing basket filter, and to sense back pressure caused by the build up of impurities in the entire system. In a further preferred embodiment, the third stage filters are 10 to 30 micron glass filters, and the fourth stage filters are a 10 to 2 micron gel filter composed of corn starch polymer embedded paper elements. In a still further preferred embodiment, the entire filtering system is designed to recirculate the biodiesel in the settling tanks, and incorporates sight glasses and sampling valves for obtaining biodiesel samples for testing prior to the biodiesel being transferred to the final distribution tank. In yet another embodiment, the filtering system is fitted with a metering system, which allows for the introduction of additives to the biodiesel in the necessary ratios.

This system allows for the custom blending of biodiesel with additives to meet the specification of a variety of end users under different climatic and operating conditions.

The interrelationship of the above components that comprise the biodiesel processing plant are illustrated in the Figures. The alcohol storage unit is connected by a suitable flow path to the mixing unit contained in the modular production unit. The triglycerides storage unit is connected by a suitable flow path to each of the feedstock settling tanks contained in the processing unit. The feedstock settling tanks, in turn, are connected by a suitable flow path to the reaction chamber.

While various preferred embodiments of the invention have been shown for purposes of illustration, it will be understood that those skilled in the art may make modifications thereof without departing from the true scope of the invention as set forth in the appended claims including equivalents thereof.

What is claimed is:

1. A biodiesel composition having a cloud point below 32° F. comprising:
a molecule according to structure I 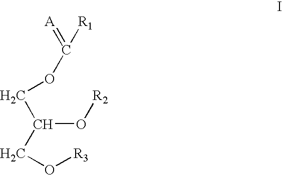
where R₁ is an alkyl chain of between 2 to 25 carbons with one or more than one double bonds and A is a hetero-atom which may be part of another moiety; and
R₂ and R₃ are independently selected from the list comprising H, C₂ to C₂₀ saturated or unsaturated alkyl, aryl, glycol, amino, hetero, heteroalkyl, heteroaryl, saturated, unsaturated, branched, straight or any combination thereof.

2. A system for a transesterification of a triglyceride below about 115° F. comprising:
combining a triglyceride and a reactant in a reaction chamber;
causing the reactant to replace a moiety on the triglyceride;
causing sound energy to pass through the reaction chamber;
extracting a post reaction mixture;
obtaining a transesterified product.

3. A method for obtaining a biodiesel product without forming a glycerin product, comprising:
causing a triglyceride and an activated nucleophile to undergo the transesterification reaction at below 115° F. forming a reaction mixture, wherein less than 3 molar equivalents of the activated nucleophile are used;
isolating the completed biodiesel product.
